(19) United States
(12) Patent Application Publication
(10) Pub. No.: US 2023/0077586 A1
(43) Pub. Date: Mar. 16, 2023

(54) Microparticle Production Platform, Method of Producing Microparticles and a Pharmaceutical Composition

(71) Applicant: Midatech Pharma (Wales) Limited, Cardiff South Glamorgan (GB)

(72) Inventors: Paul Seaman, Cardiff South Glamorgan (GB); Connor Davies, Cardiff South Glamorgan (GB); Louis King, Cardiff South Glamorgan (GB)

(21) Appl. No.: 17/801,496

(22) PCT Filed: Feb. 25, 2021
(86) PCT No.: PCT/EP2021/054758

§ 371 Date: Aug. 22, 2022

(30) Foreign Application Priority Data
Feb. 26, 2020 (GB) 2002727.2

(51) Int. Cl. B01J 2/04; B01J 2/18; A61K 9/16; A61K 38/13; A61K 38/12

(52) U.S. Cl. CPC B01J 2/04; B01J 2/18; A61K 9/1694; A61K 38/13; A61K 38/12

(57) ABSTRACT

The present invention comprises an apparatus and method for producing a microparticle and pharmaceutical compositions thereof. The apparatus and method rely on continuous inkjet (CIJ) printing to provide high quality microparticles at an improved rate.

### FIELD OF THE INVENTION

The present invention relates to a microparticle production platform, method of producing microparticles, microparticles and a pharmaceutical composition. In particular, it is directed to the production of polymeric microparticles by continuous inkjet printing.

### BACKGROUND TO THE INVENTION

A known fluid delivery system is droplet-on-demand ejection system (DOD) which uses only pressure pulses, typically generated thermally or piezoelectrically in a printing head, to dispense droplets from a nozzle.

One problem with DOD is that it requires precise negative pressure control in the printing head to prevent a syphon effect as liquid is ejected. This in itself creates a number of other disadvantages.

Firstly, a priming step is required which wastes time and around 10-25 mL of liquid in a typical array.

Secondly, there is a risk of drawing air into the DOD printer head when it is put under negative pressure such that it does not work until it is primed again.

Thirdly, to maintain a negative pressure at the DOD printing head, the liquid must be provided in a single reservoir upstream because it is not possible to use continuous flow or in-line mixing upstream of the printing head. This limits DOD to batch production and makes it difficult to handle unstable mixtures because they will need to be premixed and sit in the reservoir for prolonged periods of time.

Another problem is that the start-stop nature of DOD piezo systems creates a risk of clogging that results in the ‘drop-out’ of that system in an array.

Furthermore, DOD has an inherent maximum operating viscosity above which it is difficult to eject fluid droplets at a medium to high frequencies. Inconsistent ejection and polydisperse droplets are observed at high viscosities, which results in polydisperse microspheres.

Optimμm and Stratμm are known alternative piezo-actuated microparticle and microcapsule formation technologies. They use a two- or three-fluid vibrating nozzle configuration with an outer co-flowing axial “carrier” stream that reduces the inner jet diameter. A piezo-actuator (non-product contacting) induces droplet break-up at the nozzle outlet to form monodisperse microdroplets. The three-fluid vibrating nozzle configuration provides a coaxial inner core/API flow and an outer shell flow such that a microcapsule is formed upon ejection from the vibrating nozzle.

Optimμm is a technology platform (Orbis Biosciences, Kansas City, USA) for generating microspheres and microcapsules through a form of piezo-actuated droplet break-up. Optimμm uses a water carrier stream to make particle sizes down to 75 μm. Optimμm typically employs a coaxial laminar flow of two liquids; an inner core phase and outer shell phase. The shell phase is a hydrophobic, low-melting point wax-like material that is provided in a molten state. The shell phase may also contain a second material that is pH responsive (i.e. insoluble in water above pH 5.0). These two liquid phases are propelled by a carrier flow stream of air or nitrogen to form a jet. Optimμm is only applicable to APIs that are thermally stable due to the elevated temperature of the shell phase.

Stratμm (Orbis Biosciences, Kansas City, USA) uses a nitrogen carrier stream to make microparticle sizes down to 10 μm. Stratμm typically employs an oil/water emulsion, where a biocompatible polymer such as PLGA is dissolved in an organic, water-immiscible solvent phase. The polymer phase is ejected through a nozzle with a co-axial aqueous carrier stream. This results in liquid droplets rather than finished particles. The droplets still require hardening via solvent evaporation and subsequent lyophilisation to remove the organic solvent. Stratμm is therefore an enhanced emulsion technology that is distinct from the present invention.

There remains an unmet need for a microparticle production platform and method of producing microspheres having improved efficiency, yield, and reliability. The present invention addresses these and other needs.

### BRIEF DESCRIPTION OF THE INVENTION

Broadly, the present invention relates to an apparatus and method for producing polymeric microparticles in which one or more printing head arrangements are each configured to continuously dispense liquid droplets (the dispersed phase) into a stream of a second liquid (the continuous phase). The present inventors have surprisingly found that polymeric microparticles can be produced by continuous inkjet (CIJ) printing without compromising quality. It was initially thought that the velocities involved in CIJ printing methods would cause significant deformation of the ejected droplets, which upon rapid solvent extraction, would result in similarly misshapen polymeric microparticles. However, the configuration of present invention avoids this potential problem. The CIJ printing apparatus and method are able to handle viscous liquids without suffering from inconsistent ejection or polydisperse microspheres.

Furthermore, where an unstable liquid mixture is used (such as one including a temperature, pH, or chemically sensitive API), the CIJ printing apparatus and method allows upstream in-line mixing so that any unstable mixtures are formed immediately before processing. This means that the unstable liquid mixture exists for as short a time as possible before undergoing microparticle formation. Other advantages are that it removes the need for cooling to prevent decomposition of the unstable mixture and decreases the reliance on very high purity reagent sources. It also improves the overall quality of the final microparticles by reducing the amount of impurities.

The present inventors have found that employing CIJ provides for high frequency droplet generation, simple control systems, suitability for clean manufacture and relatively low production cost.

As described in detail herein, the or each droplet generator, operating under CIJ mode, creates a continuous stream of droplets at high frequency, by using a continuous pressure to bring the dispersed phase up to the ejection point where an acoustic wave generated by piezo crystal distortion within an applied electric field breaks the dispersed phase into a stream of droplets. The distortion of the piezo crystals causes the print nozzle to vibrate, breaking up the flow of solution into discrete microspheres through a phenomenon known as ‘Rayleigh Instability’. The continuous pressure is preferably a positive pressure. The continuous pressure may be applied to the dispersed phase by a pump, such as a reciprocating pump or a peristaltic pump. The continuous pressure may alternatively be applied by an overpressure of gas, such as nitrogen gas.

Accordingly, in a first aspect the present invention provides an apparatus for producing solid polymeric microparticles, the apparatus comprising a printing head arrangement having:
a continuous liquid droplet generator for forming liquid droplets of a first liquid by a continuous inkjet method; and
a nozzle for forming a jet of a second liquid, wherein the continuous liquid droplet generator and the nozzle are arranged relative to each other such that, in use, liquid droplets from the continuous liquid droplet generator pass through a gas into said jet of second liquid.

In some cases, the apparatus comprises an in-line mixer upstream of the continuous liquid droplet generator configured to mix two or more components to form the first liquid, such as two, three, four, five or six components. Preferably, the mixer is a static inline mixer.

In some cases, the continuous liquid droplet generator is configured to eject liquid droplets of the first liquid at a velocity of 2 m/s or more, such as 5 m/s, 10 m/s, or 20 m/s or more. Preferably, the velocity is 30 m/s or less, such as 25 m/s, 20 m/s or 15 m/s or less.

In some cases, the continuous liquid droplet generator comprises at least one piezoelectric component operable to generate droplets. The piezoelectric component may be a longitudinal actuator, shear actuator, tube actuator or contracting actuator. Preferably, the piezoelectric component is a longitudinal actuator. Preferably, the piezoelectric component is provided in a chamber having a micron-sized orifice through which droplets generated by the piezoelectric component may be ejected.

In some cases, the piezoelectric component is configured to generate an acoustic wave by piezo crystal distortion within an applied electric field such that the nozzle vibrates and the continuous flow is broken up into discrete droplets by a phenomenon known as ‘Rayleigh Instability’.

In some cases, the apparatus comprises a signal generator operable to supply an electric field to the piezoelectric component.

In some cases, the piezoelectric component comprises a heater configured not to exceed 55° C. The heater may be an electric heater. Alternatively, the heater may be a heating block that is either solid or filled with a thermally conductive fluid. The heater may be configured not to exceed 50° C., such as 45° C., 40° C. or 35° C. The heater may be configured to exceed 35° C., such as 40° C., 45° C. or 50° C. A heater is useful when handling viscous liquids. To handle most viscous liquids, a single nozzle DOD piezo head requires an operating temperature of >70° C. and a DOD piezo array requires about >60° C., but the present CIJ printing apparatus can operate without problems at 50° C. or less. This reduces the thermal load on the first liquid and mitigates the loss of value API material by side reactions or decomposition. Where there are a plurality of printing head arrangements in a row, a heater may extend across all the printing heads, such as in the form of a heating rod or a rail. An advantage of this is that consistent heating across all the printing heads can be achieved and individual heaters do not need to be maintained in each printing head arrangement.

In some cases, the heater is contained inside the piezoelectric component such that, when in use, it does not directly contact the first liquid. Unlike the heater in DOD piezo systems, the present heater is not in direct contact with the fluid path and so it does not need to be sterilised. Sterilisation of the fluid-contacting heaters in DOD systems cannot be done by autoclaving or gamma irradiation as they may be damaged by the process conditions, due to complex internal electronic components. The use of non-conventional sterilisation techniques, such as E-beam sterilisation, is often required for reliable sterilisation. The present CIJ apparatus avoids this sterilisation problem.

In some cases, the continuous liquid droplet generator is in the form of an inkjet printhead. That is to say, the continuous liquid droplet generator may be provided in a self-contained body that is easy to handle. It may be modular for straightforward connection to and removal from the apparatus. It may be ‘plug-and-play’ such that no further configuration is necessary after connection to the apparatus.

In some cases, the continuous liquid droplet generator and nozzle are arranged such that, in use, the liquid droplets of a first liquid and the jet of a second liquid meet at an angle greater than 0° and less than 90° (i.e. an acute angle). Preferably, the angle is greater than 10° and less than 80°, such as greater than 20° and less than 70° or greater than 30° and less than 60°. By having a contact angle between the first liquid and the second liquid that is less than 90 degrees, the deformation of the droplets as they impact the anti-solvent stream is reduced, therefore it is not necessary to reduce ejection velocity or throughput rate. This advantage is more pronounced the closer the angle is to 0°. The ejection velocity may therefore be higher the closer the angle is to 0°.

In some cases, the continuous liquid droplet generator is operable to generate liquid droplets having an individual droplet volume in the range 1 to 100 pL, optionally in the range 5 to 50 pL, such as 39 to 45 pL, preferably 42 pL.

In some cases, the continuous liquid droplet generator is operable to produce liquid droplets at a frequency of more than 100 kHz, such as 110 to 500 kHz, 120 to 250 kHz or 130 to 150 kHz. In one particular case, the liquid droplet generator is operable to produce liquid droplets at a frequency of 128 kHz.

In some cases, the apparatus further comprises a microparticle-receiving means for receiving solid microparticles dispersed in a jet of liquid. In particular, the microparticle-receiving means may comprise a conduit having an opening arranged such that, in use, the jet of second liquid enters said opening downstream of the region of the jet where the liquid droplets enter the jet of second liquid. In some cases, the microparticle-receiving means comprises a tube having an opening that faces said nozzle. The tube may be formed of flexible or rigid material and may comprise an elbow bend. Typically, the microparticle-receiving means is able to convert the generally horizontal motion of the microparticle-containing jet into downward vertical motion for collection of the microparticles and/or separation of the microparticles from the second liquid.

In some cases, the microparticle-receiving means comprises a fluid removal means operable to remove fluid from the microparticle-receiving means and a microparticle collection means operable to remove microparticles from the microparticle-receiving means.

In some cases, the apparatus further comprises means for generating a flow of said second liquid through said nozzle. In particular, the means for generating flow may comprise a regulated pressure system for producing a pulseless flow of the liquid. In certain cases, the means for generating flow may comprise a reservoir for holding the second liquid, said reservoir having an outlet in fluid communication with said nozzle.

In some cases, the nozzle has a reduction in cross-sectional area in the direction of flow so as to increase the flow velocity of a liquid passing through the nozzle and thereby form a jet.

In some cases, the apparatus further comprises a camera for monitoring liquid droplets generated by said continuous liquid droplet generator. Alternatively or additionally, the apparatus may further comprise a light source for illuminating liquid droplets generated by said continuous liquid droplet generator. In particular, the light source may comprise an LED strobe electrically coordinated with the continuous liquid droplet generator such that, in use, the camera is able to capture an image of liquid droplets ejected from the continuous liquid droplet generator at a pre-determined (but typically user-adjustable) time period after ejection of said liquid droplets. For example, the LED strobe may have an adjustable strobe delay, adjustable strobe intensity and/or adjustable pulse width settings, thereby allowing said pre-determined time period after ejection of said droplets to be adjusted.

In some cases, the apparatus further comprises at least one temperature regulator for controlling the temperature of liquid entering said continuous liquid droplet generator and/or the temperature of liquid entering said nozzle. In particular, the at least one temperature regulator may comprise a first chiller for controlling the temperature of the first liquid entering the continuous liquid droplet generator in the range of 5° C. to 30° C., optionally in the range 12° C. to 16° C. or 16° C. to 20° C. In certain cases, the at least one temperature regulator comprises a second chiller for controlling the temperature of the second liquid entering the nozzle in the range of 0° C. to 20° C., optionally in the range 2° C. to 8° C. or 3° C. to 9° C.

The nozzle may be arranged such that, in use, the jet is directed laterally so as to define a horizontal line or arc that passes below the liquid droplet generator. In particular, the liquid droplet generator may be arranged such that, in use, the liquid droplets are ejected downwardly with an initial velocity and/or under the assistance of gravity, through said gas, into said jet of second liquid. Alternatively, the nozzle and liquid droplet generator may be arranged such that the jet of the nozzle and the stream of liquid droplets are both ejected substantially laterally through the gas such that they combine at a predefined point.

In some cases, the continuous liquid droplet generator is positioned relative to the nozzle such that the distance of travel of a liquid droplet from the continuous liquid droplet generator to the nearest point of the jet is in the range 2 to 10 mm, optionally 4 to 6 mm.

In some cases, there is a plurality of printing head arrangements. The number of printing head arrangements is 2 to 1000, such as 5 to 100, 10 to 50 or 20 to 30. Each printing may be configured in the same way. Alternatively, some or all of the printing heads may be separately and individually configured.

In some cases, the nozzles of the plurality of printing head arrangements are spaced-apart at equal intervals. In particular, the nozzles of adjacent liquid droplet generators may be spaced-apart by between 5 and 25 mm, measured nozzle centre to nozzle centre, such as between 10 and 20 mm.

In some instances, the plurality of printing head arrangements are arranged in parallel such that each of the liquid droplets are ejected in parallel and each of the jets are provided in parallel. In other instances, the plurality of printing head arrangements are aligned or staggered.

In a second aspect, the present invention provides a process for producing solid microparticles, the process comprising:
providing a first liquid comprising a solute and a solvent, the solute comprising a biocompatible polymer, the concentration of polymer in the first liquid optionally being at least 10% w/v, ‘w’ being the weight of the polymer and ‘v’ being the volume of the solvent,
providing a continuous liquid droplet generator operable to generate liquid droplets,
providing a jet of a second liquid,
causing the continuous liquid droplet generator to form liquid droplets of the first liquid,
passing the liquid droplets through a gas to contact the jet of the second liquid so as to cause the solvent to exit the droplets, thus forming solid microparticles,
the solubility of the solvent in the second liquid being at least 5 g of solvent per 100 mL of second liquid, the solvent being substantially miscible with the second liquid.

Preferred parameters of the process include one or more of a droplet velocity of 10 to 14 m/s, such as 12 m/s; a droplet volume of 39 to 45 pL, such as 42 pL; a polymer feeding pressure of between 6 to 8 bar, such as 7 bar; and a jet velocity that is 1.1 to 1.3 times the liquid droplet velocity, such as 1.2 times.

In some cases, the first liquid is a mixture that is prepared upstream of the liquid droplet generators by in-line mixing. In-line mixing minimises the amount of time a mixture is held before microsphere formation. An advantage is that unstable mixtures may be handled with minimal decomposition. Additionally, the point of in-line mixing may be positioned in close proximity to the continuous liquid droplet generator to further minimise time for cross-reaction or decomposition.

In some instances, the first liquid comprises two components having a reaction half-life of two hours or less at standard temperature and pressure. The reaction half-life may be one hour or less, thirty minutes or less or 10 minutes or less. The two components may be the solute and the solvent. Alternatively, the two components may each be a solute in the solvent. There may be cases where complex multicomponent systems of three or more components also undergo complex unwanted side-reactions.

In some cases, the first liquid further comprises at least one (e.g. 1, 2, 3, 4, 5 or more different target materials) target material (also known as a “payload”) which is desired to be encapsulated within the microparticles, the target material being incorporated in the first liquid as a particulate or in solution. Preferably, the target material is in solution. In certain cases, the target material comprises a pharmaceutically active agent or a precursor of a pharmaceutically active agent. In particular, the target material may be a pharmaceutically active agent or a precursor of a pharmaceutically active agent for treatment of a tumour, a central nervous system (CNS) condition, an ocular condition, an infection (e.g. viral, bacterial or other pathogen) or an inflammatory condition (including autoinflammatory conditions).

In some cases, the target material may be a peptide, a hormone therapeutic, a chemotherapeutic or an immunosuppressant. In particular, the target material may comprise octreotide or a salt thereof (e.g. octreotide acetate), or ciclosporin A or a salt thereof.

In some cases, the target material may comprise a plurality of nanoparticles. In particular, the nanoparticles may have a pharmaceutically active agent or a precursor of a pharmaceutically active agent covalently or non-covalently (e.g. electrostatically) bound thereto (directly or via one or more linkers). The nanoparticles may, for example, be as described in PCT/EP2015/076364 filed 11 Nov. 2015, published as WO 2016/075211 A1—the entire contents of which is expressly incorporated herein by reference).

In some cases, the continuous liquid droplet generator comprises at least one piezoelectric component operable to generate droplets.

In some cases, the piezoelectric component may be a longitudinal actuator, shear actuator, tube actuator or contracting actuator. Preferably, the piezoelectric component is a longitudinal actuator.

In some cases, the piezoelectric component is configured to generate an acoustic wave by piezo crystal distortion within an applied electric field such that the nozzle vibrates and the continuous flow is broken up into discrete droplets by a phenomenon known as ‘Rayleigh Instability’.

In some cases, the number of liquid droplet generator outlets is in the range 5 to 150, such as 10 to 80, 20 to 70 or 30 to 60.

In some cases, the frequency of liquid droplet generation is of more than 100 kHz, such as 110 to 500 kHz, 120 to 250 kHz or 130 to 150 kHz. In one particular case, the liquid droplet generator is operable to produce liquid droplets at a frequency of 128 kHz. Assuming a droplet size of 42 pL and 120 kHz frequency, each continuous print head delivers 5 μL/nozzle/sec of liquid droplets to the anti-solvent and therefore a set-up of 20 printing head arrangements would process 2.88 L in a typical run time of 8 hours.

In some cases, the liquid droplets have an individual droplet volume in the range 1 to 100 pL, optionally 20 to 60 pL.

In some cases, the mean greatest dimension (typically the diameter) of the solid microparticles is in the range 1 to 200 μm, optionally 10 to 100 μm or 15 to 25 μm or 20 to 40 μm.

In some cases, the coefficient of variation of the greatest dimension of the microparticles is 0.1 or less, the coefficient of variation being the standard deviation of the greatest dimension of the microparticles divided by the mean greatest dimension. The present inventors have found that despite the increased production scale of the method of present invention, the resulting microparticles exhibit excellent uniformity of size and shape, i.e. they form a substantially monodisperse population.

In some cases, the ratio of the greatest dimension to the least dimension of the microparticles is in the range 2 to 1, optionally 1.1 to 1.01. In particular, the microparticles may be substantially spherical (“microspheres”).

In some cases, the jet of second liquid is generated by providing a continuous, pulseless flow of said second liquid and passing said flow of second liquid through a nozzle which causes a reduction in the cross-sectional area available for flow and thereby increases the flow velocity of the second liquid, said nozzle terminating in an orifice from which the jet of second liquid emerges.

In some cases, the jet of second liquid passes through a gas (e.g. air).

In some cases, the jet of second liquid is not in contact with any wall or channel for at least part of its length. This differs from prior-described methods, in which the continuous phase is generally provided as a flow in a channel or a pool such as a stirred pool in an open-topped vessel. In particular embodiments, the part of the length of the jet not in contact with any wall or channel comprises a contact zone, said contact zone being the zone of the jet in which said liquid droplets make contact with said jet. In particular embodiments, the part of the length of the jet not in contact with any wall or channel comprises the length from the nozzle up to and including the contact zone.

In some cases, the liquid droplets pass through gas (e.g. air) for a distance of less than 25 mm, 10 mm or 5 mm and optionally more than 1 mm, 2 mm, 3 mm or 5 mm before contacting said jet of second liquid.

In some cases, the jet of second liquid flows substantially at an angle greater than 0° and less than 90° (i.e. an acute angle) to the direction of droplet ejection. Preferably, the angle is greater than 10° and less than 80°, such as greater than 20° and less than 70° or greater than 30° and less than 60°.

In some cases, the continuous liquid droplet generator is positioned above the jet of second liquid and said liquid droplets are ejected downwards towards the jet of second liquid.

In some cases, the continuous liquid droplet generator dispenses liquid droplets from their respective outlets simultaneously. In particular, the liquid droplets may pass through gas in parallel before contacting said jet of second liquid.

In some cases, the flow velocity of the jet of second liquid and the frequency of liquid droplet generation are selected such that the liquid droplets and/or the solid microparticles do not coalesce. In particular, the flow rate of the jet of the second liquid may be in the range 10 to 500 mL/min, such as 20 to 200 mL/min or 20 to 100 mL/min.

In some cases, the process is carried out under aseptic conditions, optionally within a laminar flow cabinet. This is particularly suitable when the target material is a pharmaceutical and/or when the microparticles are intended for therapeutic or other clinical use. In the case where the process is carried out in a laminar flow cabinet, the relative position of the continuous liquid droplet generator and the jet of the second liquid may be chosen to account for the direction and speed of air flow of the laminar flow cabinet, thereby causing the liquid droplets to contact the jet of the second liquid.

In some cases, the process of the invention further comprises capturing one or more images of at least one of said liquid droplets at a pre-determined time point after the at least one liquid droplet has been generated. In particular, the process may further comprise deriving from said one or more images at least one liquid droplet property selected from the group consisting of: droplet velocity, droplet volume, droplet radius and deviation of droplet from its initial trajectory. In this way, monitoring (including continuous live monitoring) of droplet properties can be integrated or fed back to adjust, if necessary, one or more process parameters such as droplet generation frequency, the flow rate of the jet of second liquid or the temperature of the first and/or second liquids in order to control the size and other properties of the microparticles produced.

In some cases, the process of the invention includes using process analytical technology (PAT). The process may comprise taking an in-line measurement by spectroscopy and/or mass spectrometry. The measurement may be of one or more of the liquid droplets, the microparticles, the first liquid and the second liquid. The spectrometer, optical or sample-probe may be positioned in proximity to the point of measurement.

In some cases, the temperature of the first liquid entering the continuous liquid droplet generator is in the range of 5° C. to 30° C., optionally in the range 12° C. to 16° C. or 16° C. to 20° C.

In some cases, the temperature of the second liquid entering the nozzle is in the range of 0° C. to 20° C., optionally in the range 2° C. to 8° C. or 3° C. to 9° C.

In some cases, the solvent is a biocompatible solvent. The solvent may be a class III solvent (United States Pharmacopoeia 467). The solvent may be one or more of dimethyl sulfoxide (DMSO), n-methyl pyrrolidone, hexafluoro-isopropanol, glycofurol, propylene carbonate, dimethyl isosorbide, cyrene, 2,2,5,5-tetramethyloxolane, triacetin, PEG200 and PEG400.

In some cases, the second liquid comprises a mixture of water and an alcohol (e.g. tert-butanol) or water and a water-soluble organic compound other than an alcohol, optionally at 10% to 20% v/v water to alcohol or water-soluble organic compound. In particular, the second liquid may be 10 to 20%, such as 15% v/v, tertiary butanol in water.

In some cases, the polymer comprises a poly(lactide), a poly(glycolide), a polycaprolactone, a polyanhydride and/or a co-polymer of lactic acid and glycolic acid, or is any combination of said polymers or co-polymers. In particular, the polymer may comprise Resomer RG752H, Purasorb PDL 02A, Purasorb PDL 02, Purasorb PDL 04, Purasorb PDL 04A, Purasorb PDL 05, Purasorb PDL 05A Purasorb PDL 20, Purasorb PDL 20A; Purasorb PG 20; Purasorb PDLG 5004, Purasorb PDLG 5002, Purasorb PDLG 7502, Purasorb PDLG 5004A, Purasorb PDLG 5002A, Resomer RG755S, Resomer RG503, Resomer RG502, Resomer RG503H, Resomer RG502H, Resomer RG752, or any combination thereof.

In some cases, the process further comprises a step of collecting the solid microparticles by separating the solid microparticles from the second liquid. In particular, the process may further comprise subjecting the microparticles to one or more post-production treatment steps selected from the group consisting of: washing, heating, drying, freeze-drying and sterilizing.

In some cases, the process further comprises formulating or packaging the microparticles into a pharmaceutical composition or delivery form. For example, the microparticles may be combined with a pharmaceutically acceptable carrier, diluent or vehicle. In some embodiments the pharmaceutical composition or delivery form may be a depot injection.

In some cases, the process of the second aspect of the invention employs an apparatus in accordance with the first aspect of the invention.

In a third aspect, the present invention provides a microparticle produced or producible by the process of the second aspect of the invention.

In a fourth aspect, the present invention provides a pharmaceutical composition comprising a microparticle of the third aspect of the invention and a pharmaceutically acceptable carrier, diluent, excipient, salt and/or solution.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 shows a schematic diagram of a fluid delivery skid having a static in-line mixing point for combining the active and polymer phases to form the dispersed phase.

FIG. 2 shows a side view a single printing head arrangement in use with the jet and liquid droplets meeting at a set angle of incidence (θ).

FIG. 3 shows a perspective view of a housed single printing head arrangement in use with the jet and liquid droplets meeting at a set angle of incidence (θ).

FIG. 4 shows a perspective view of 10 housed printing head arrangements provided in parallel, in a frame and in use such that the liquid droplets are generated in parallel and the jets are provided in parallel.

FIG. 5 shows a section view of a printing head arrangement having a drive rod that may be actuated to form droplets.

### DETAILED DESCRIPTION OF THE INVENTION

In describing the present invention, the following terms will be employed, and are intended to be defined as indicated below.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The following is presented by way of example and is not to be construed as a limitation to the scope of the claims.

In describing the present invention, the following terms will be employed, and are intended to be defined as indicated below.

Microparticles

Microparticles in accordance with the present invention may be in the form of solid beads. As used herein in connection with microparticles or beads, solid is intended to encompass a gel. Microparticles as used herein specifically include any polymeric particle or bead of micron scale (typically from 1 μm up to 999 μm in diameter). The microparticles may be of substantially spherical geometry (also referred to herein as “microspheres”). In particular, the ratio of the longest dimension to the shortest dimension of the microparticle may be not more than 5, 4, 3, 2, 1.9, 1.8, 1.7, 1.6, 1.5, 1.4, 1.3, 1.2, 1.1, 1.05 or not more than 1.01.

Jet

As used herein, a “jet” is a coherent stream of fluid that is projected into a surrounding medium from a nozzle or aperture. In particular, a jet of second liquid (continuous phase) may be a coherent stream of the second liquid projected into a gas (typically air) from a nozzle. The jet may define a flow path, at least part of which is not in contact with any solid wall, conduit or channel. The jet may define a flow path (e.g. a line or arc) that intersects with the path or paths of liquid droplets dispensed from the continuous droplet generator. For example, the jet may be a stream of the second liquid passing through air below the continuous droplet generator, whereby liquid droplets dispensed from the droplet generator pass through the gas under the assistance of gravity into the stream of the second liquid and are carried by said stream of second liquid. Typically, surface tension of the second liquid contributes to the jet taking the form of coherent stream. In some cases, the jet has a substantially circular cross-section. However, other cross sectional shapes (e.g. flattened or oval-like) are specifically contemplated and may be provided, e.g., by means of particular nozzle shapes.

Biocompatible Polymer

The polymer is typically a biocompatible polymer. “Biocompatible” is typically taken to mean compatible with living cells, tissues, organs, or systems, and posing minimal or no risk of injury, toxicity, or rejection by the immune system. Examples of polymers which may be used are polylactides (with a variety of end groups), such as Purasorb PDL 02A, Purasorb PDL 02, Purasorb PDL 04, Purasorb PDL 04A, Purasorb PDL 05, Purasorb PDL 05A Purasorb PDL 20, Purasorb PDL 20A; polyglycolides (with a variety of end groups), such as Purasorb PG 20; polycaprolactones; polyanhydrides, and copolymers of lactic acid and glycolic acid (with a variety of end groups, L:G ratios and molecular weight can be included), such as Purasorb PDLG 5004, Purasorb PDLG 5002, Purasorb PDLG 7502, Purasorb PDLG 5004A, Purasorb PDLG 5002A, resomer RG755S, Resomer RG503, Resomer RG502, Resomer RG503H, Resomer RG502H, RG752, RG752H, or combinations thereof. In some cases, it is preferred that the solute is substantially insoluble in water (it is convenient to use water as the second liquid). If the second liquid comprises water, it is preferred that the solvent is a water-miscible organic solvent, such as dimethyl sulfoxide (DMSO), n-methyl pyrrolidone, hexafluoro-isopropanol, glycofurol, propylene carbonate, dimethyl isosorbide, cyrene, PEG200 and PEG400.

The weight average molecular weight (MW) of the polymer may be from 4 to 700 kDaltons, particularly if the polymer comprises a poly (α-hydroxy) acid. If the polymer comprises a copolymer of lactic and glycolic acid (often called “PLGA”), said polymer may have a weight average molecular weight of from 4 to 120 kDaltons, preferably of from 4 to 15 kDaltons.

If the polymer comprises a polylactide, said polymer may have a weight average molecular weight of from 4 to 700 kDaltons.

The polymer may have an inherent viscosity of from 0.1-2 dl/g, particularly if the polymer comprises a poly (α-hydroxy) acid. If the polymer comprises a copolymer of lactic and glycolic acid (often called “PLGA”), said polymer may have an inherent viscosity of from 0.1 to 1 dl/g, and optionally of from 0.14 to 0.22 dl/g. If the polymer comprises a polylactide, said polymer may have an inherent viscosity of from 0.1 to 2 dl/g, and optionally of from 0.15 to 0.25 dl/g. If the polymer comprises a polyglycolide, said polymer may have an inherent viscosity of from 0.1 to 2 dl/g, and optionally of from 1.0 to 1.6 dl/g. It is preferred that the first liquid comprises a target material which is desired to be encapsulated within the solid microparticles. However, it is specifically contemplated herein that the process of the present invention may, in certain cases, not include a target material. For example, the process may be used to produce placebo microparticles, e.g., for use as a negative control in an experiment or clinical trial.

Target Material

The target material (also known as the “payload”) may be incorporated in the first liquid as a particulate or may be dissolved. The target material may comprise a pharmaceutically active agent, or may be a precursor of a pharmaceutically active agent. In some cases, the target material comprises a pharmaceutically active agent, or precursor (e.g. prodrug) thereof, for treatment of a tumour, a central nervous system (CNS) condition, an ocular condition, an infection or an inflammatory condition. In some cases, the target material may comprise a peptide, a hormone therapeutic, a chemotherapeutic or an immunosuppressant. In certain cases, said target material comprises a plurality of nanoparticles (e.g. gold nanoparticles). When present, such nanoparticles may have a pharmaceutically active agent or a precursor thereof covalently or non-covalently bound thereto.

Examples of pharmaceutically active agent include, for example, any agent that is suitable for parenteral delivery, including, without limitation, fertility drugs, hormone therapeutics, protein therapeutics, anti-infectives, antibiotics, antifungals, cancer drugs, pain-killers, anti-emetics, vaccines, CNS drugs, and immunosupressants. Particular examples include octreotide or salt thereof (e.g. octreotide acetate) and ciclosporin A or a salt thereof.

The delivery of drugs in polymer microparticles, especially by controlled release parenteral, intravitreal or intracranial delivery, has particular advantages in the case of drugs which, for example, have poor water-solubility, high toxicity, poor absorption characteristics, although the invention is not limited to use with such agents. The active agent may be, for example, a small molecular drug, or a more complex molecule such as a polymeric molecule. The pharmaceutically active agent may comprise a peptide agent. The term “peptide agent” includes poly(amino acids), often referred to generally as “peptides”, “oligopeptides”, “polypeptides” and “proteins”. The term also includes peptide agent analogues, derivatives, acylated derivatives, glycosylated derivatives, pegylated derivatives, fusion proteins and the like. Peptide agents which may be used in the method of the present invention include (but are not limited to) enzymes, cytokines, antibodies, vaccines, growth hormones and growth factors.

The target material (especially in the case of a pharmaceutically active agent or a precursor thereof) may be provided in an amount of 2-70% w/w compared to the weight of the polymer, optionally from 5 to 40% w/w, further optionally from 5 to 30% w/w and more optionally from 5-15% w/w.

If the target material comprises a peptide agent, the first liquid may comprise one or more tertiary structure alteration inhibitors. Examples of tertiary structure alteration inhibitors are: saccharides, compounds comprising saccharide moieties, polyols (such as glycol, mannitol, lactitol and sorbitol), solid or dissolved buffering agents (such as calcium carbonate and magnesium carbonate) and metal salts (such as CaCl₂, MnCl₂, NaCl and NiCl₂). The first liquid may comprise up to 25% w/w tertiary structure alteration inhibitors, the weight percentage of the tertiary structure alteration inhibitor being calculated as a percentage of the weight of the polymer. For example, the first liquid may comprise from 0.1 to 10% w/w (optionally from 1 to 8% w/w and further optionally from 3 to 7% w/w) metal salt and 0.1 to 15% w/w (optionally from 0.5 to 6% w/w and further optionally from 1 to 4% w/w) polyol.

Second Liquid

The second liquid (also referred to herein as the “continuous phase”) may comprise any liquid in which the solute (typically a polymer) is substantially insoluble. Such a liquid is sometimes referred to as an “anti-solvent”. Suitable liquids may include, for example, water, methanol, ethanol, propanol (e.g. 1-propanol, 2-propanol), butanol (e.g. 1-butanol, 2-butanol or tert-butanol), pentanol, hexanol, heptanol, octanol and higher alcohols; diethyl ether, methyl tert butyl ether, dimethyl ether, dibutyl ether, simple hydrocarbons, including pentane, cyclopentane, hexane, cyclohexane, heptane, cycloheptane, octane, cyclooctane and higher hydrocarbons. If desired, a mixture of liquids may be used.

The second liquid preferably comprises water, optionally with one or more surface active agents, for example, alcohols, such as methanol, ethanol, propanol (e.g. 1-propanol, 2-propanol), butanol (e.g. 1-butanol, 2-butanol or tert-butanol), isopropyl alcohol, Polysorbate 20, Polysorbate 40, Polysorbate 60, Polysorbate 80, polyethylene glycols and polypropylene glycols. Surface active agents, such as alcohols, reduce the surface tension of the second liquid receiving the droplets, which reduces the deformation of the droplets when they impact the second liquid, —thus decreasing the likelihood of non-spherical droplets forming. This is particularly important when the extraction of solvent from the droplet is rapid. If the second liquid comprises water and one or more surface active agents, the second liquid may comprise a surface active agent content of from 1 to 95% v/v, optionally from 1 to 30% v/v, optionally from 1 to 25% v/v, further optionally from 5% to 20% v/v and further more optionally from 10 to 20% v/v. The % volume of surface active agent is calculated relative to the volume of the second liquid.

The entire contents of WO2012/042274, WO 2012/042273 and WO 2013/014466 are expressly incorporated herein by reference for all purposes.

The following is presented by way of example and is not to be construed as a limitation to the scope of the claims.

Microsphere Manufacturing Apparatus and Process

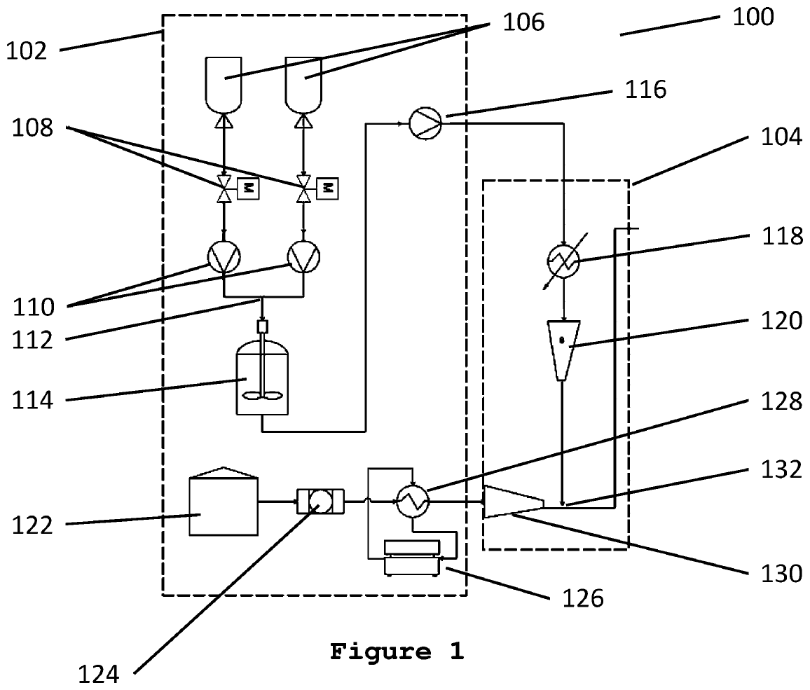

FIG. 1 shows a schematic diagram 100 of the present microsphere manufacturing apparatus that includes a fluid delivery skid 102 having a static in-line mixer 114 for combining the active and polymer phases to form the first liquid and providing the second liquid. There is also a microsphere generation skid 104 for forming uniformly-sized resorbable polymer microspheres by a de-solvation method.

The fluid delivery skid 102 has two collapsible, bottom-feeding inert bags 106, one holding a polymer phase and the other holding an active phase. The delivery of each phase is controlled by a dedicated motorised valve 108 and is pumped by a dedicated low pressure pump 110 before combination at point 112 and subsequent static in-line mixing in mixing vessel 114. The first liquid is thereby formed as a single homogenous phase. In-line mixing occurs within only a few seconds of the phases entering the system. A high pressure pump 116 transfers the first liquid from the fluid delivery skid 102 to the microsphere generation skid 104.

In another part of the fluid delivery skid 102 the second liquid is delivered from a pressure vessel 122 through a filter 124 and a heat exchanger and chiller 128.

In microsphere generation skid 104, the first liquid is heated by heater 118 and ejected via droplet nozzle 120. Similarly, the second liquid is ejected through jet nozzle 130. The first and second liquid then separately pass through a gas and combine at pre-determined point 132 where the microspheres are then formed by a desolvation mechanism. The stream of residual combined liquid carrying the generated microspheres then proceeds sequentially to a dewatering skid and a washing skid (not shown).

In the dewatering skid, the microspheres are separated from the combined liquid stream in a rotating sieve. A vacuum is drawn from underneath the sieve that has a pore size smaller than the microspheres, which aids in the removal of the waste fluid from the suspension. The resulting ‘dried’ microspheres are then entrained within a flow of air and captured by means of a cyclonic separator. During the conveying stage, moisture from the surface of the microparticles evaporates thereby reducing the moisture content further. The cyclone separates the powder from the conveying airflow and the dewatered microspheres are collected within a powder vessel underneath the cyclone. Liquid removed from the suspension is collected into a waste vessel for subsequent disposal.

In the washing skid, the solid microspheres are washed in a specific medium, at controlled temperatures and for a set length of time. A first wash is conducted with a solution of D-Mannitol, a type of sugar alcohol, which strips away and dissolves any API on the surface of the microparticles during the wash. The washing of the microparticles removes surface-bound API and also confers a level of polymer remodelling or ‘healing’. This healing of the microparticle provides a closed and intact surface that affects the rate at which water can enter the microparticles, and therefore affects the API dissolution profile. A ‘jacket’ on the mixing vessel allows for heating and cooling of the wash media. A recipe or procedure is programmed into the heat exchanger which automates the heating and cooling of the wash solution as required. A powder induction mixer is used to induce powders below the surface of the fluid, causing immediate wetting below the surface of the liquid and avoiding agglomeration and/or adhesion of the microspheres to each other, the wall of the vessel or any installed components. Mannitol is removed from the wash vessel at the end of the first wash cycle by tangential flow filtration. Water is added at the same rate of removal to keep the product suspended. A second wash is then initiated by adding a concentrated solution of phosphate buffered saline (PBS) to the vessel. At the end of the PBS cycle, the product is pumped again to a dewatering skid. A spray ball is inserted into the roof of the wash vessel to rinse down the vessel surfaces during the discharge phase with a small amount of water and acts to remove product that has adhered onto the vessel surfaces, to increase product recovery. The product may then be filled in vials, lyophilised, stoppered and capped.

Printing Head Arrangement

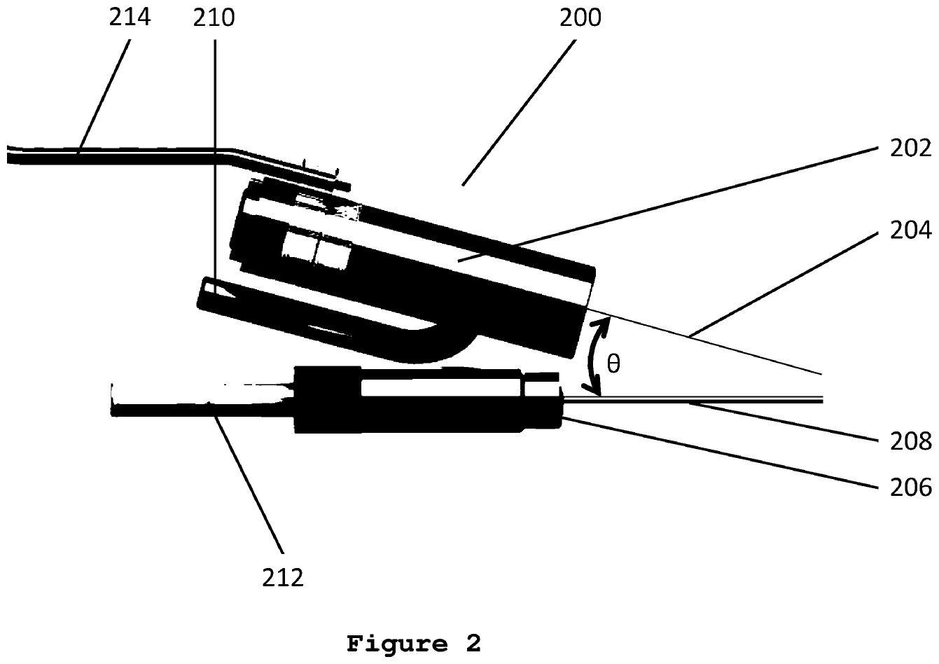

FIG. 2 shows one case where there is provided an apparatus 200 that is a printing head arrangement having a continuous droplet generator 202 that provides a first liquid 204 by a continuous inkjet method. There is also a nozzle 206 providing a jet of a second liquid 208. The first liquid 204 and second liquid 208 combine at a point to the right beyond the boundary of the figure. The continuous liquid droplet generator 202 is supported on an arm 214 and has a fluid inlet 210. The nozzle 206 also has a fluid inlet 212 for supplying the second liquid 208. The liquid droplet generator 202 and nozzle 206 are arranged such that the stream of the liquid droplets 204 and the jet 208 are both ejected substantially laterally through the gas such that they combine at a predefined point (not shown).

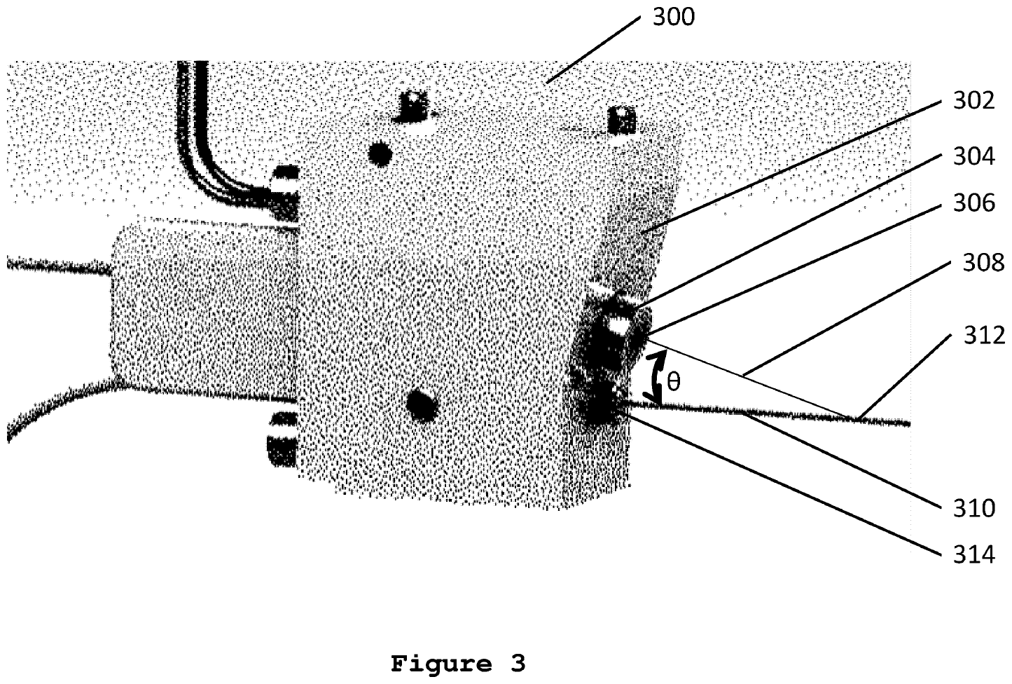

FIG. 3 shows another case where the apparatus 300 is a printing head arrangement provided with an enclosing body 302 that is easy to handle and use as a module in a larger modular apparatus. The tips of the continuous droplet generator 304 and nozzle 314 each protrude from a corresponding hole in the body 302 such that the first liquid 308 and second liquid 310 are ejected clear of the body 302. The liquid droplet generator 304 and nozzle 314 are arranged such that the stream of the liquid droplets 308 and the jet 310 are both ejected substantially laterally through the gas such that they combine at a predefined point (312).

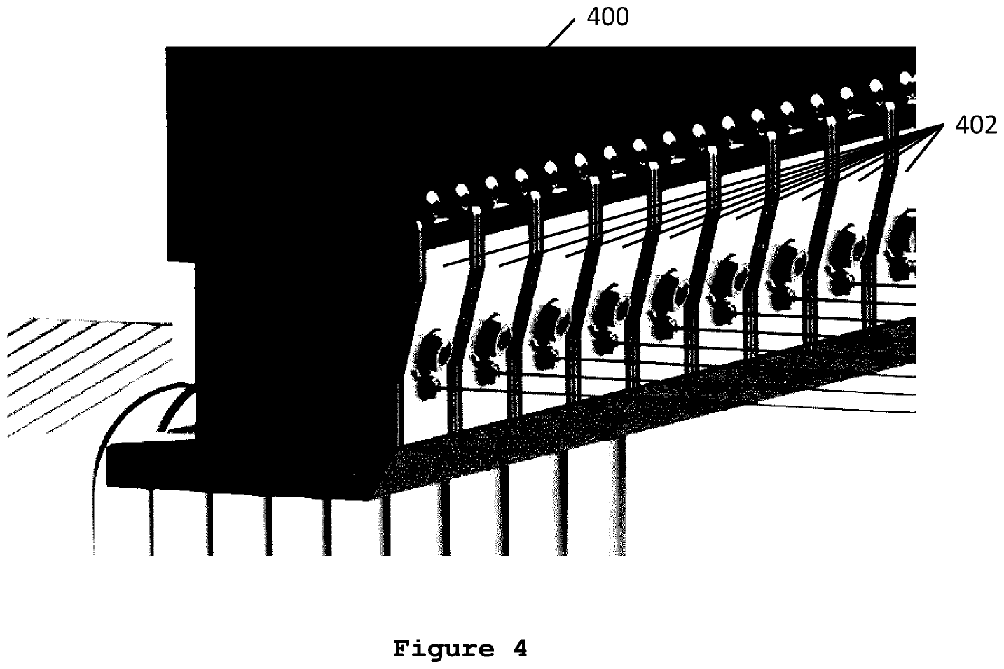

FIG. 4 shows another case where the apparatus 400 comprises a frame containing a plurality of printing head arrangements 402 that are arrange in a row, equidistant from one another, and ejecting their first and second liquids in parallel.

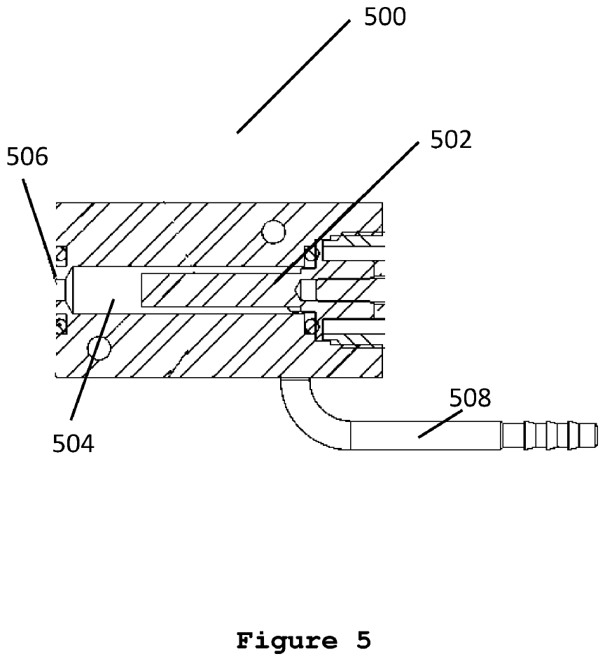

FIG. 5 shows one instance where the continuous liquid droplet generator 500 of a printing head arrangement has a drive rod (a longitudinal actuator) 502 that occupies space in an ejection chamber 504. When the first liquid is delivered under pressure to the ejection chamber 504 via the inlet 508, the drive rod 502 is actuated to form droplets from the first liquid that are then ejected out of micron sized outlet 506 by displacement by more first liquid entering the chamber 504.

### oOo

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only and are not to be construed as limiting the disclosure in any way.

1. An apparatus for producing solid polymeric microparticles, the apparatus comprising a printing head arrangement having:
a continuous liquid droplet generator for forming liquid droplets of a first liquid by a continuous inkjet method; and
a nozzle for forming a jet of a second liquid,
wherein the liquid droplet generator and the nozzle are arranged relative to each other such that, in use, liquid droplets from liquid droplet generator pass through a gas into said jet of second liquid.

2. An apparatus according to claim 1 wherein the apparatus comprises an in-line mixer upstream of the continuous liquid droplet generator for mixing two or more components to form the first liquid.

3. An apparatus according to any one of the preceding claims wherein the continuous liquid droplet generator is configured to eject liquid droplets of the first liquid at a velocity of 2 m/s or more.

4. An apparatus according to any one of the preceding claims wherein the continuous liquid droplet generator comprises a piezoelectric component operable to generate droplets.

5. An apparatus according to claim 4 wherein the piezoelectric component is configured to generate an acoustic wave by piezo crystal distortion within an applied electric field such that the nozzle vibrates and the continuous flow is broken up into discrete droplets by a phenomenon known as ‘Rayleigh Instability’.

6. The apparatus according to claim 4 or claim 5, further comprising a signal generator operable to supply an electric field to the piezoelectric component.

7. An apparatus according to any one of claims 4 to 6 wherein the piezoelectric component comprises a heater configured not to exceed 55° C.

8. An apparatus according to claim 7 wherein the heater is contained inside the piezoelectric component such that, when in use, it does not directly contact the first liquid.

9. The apparatus according to any one of the preceding claims, wherein the continuous liquid droplet generator is in the form of an inkjet printhead.

10. The apparatus according to any one of the preceding claims wherein the continuous liquid droplet generator and nozzle are arranged such that, in use, the liquid droplets of a first liquid and the jet of a second liquid meet at an angle greater than 0° and less than 90°.

11. The apparatus according to any one of the preceding claims, wherein the continuous liquid droplet generator is operable to generate liquid droplets having an individual droplet volume in the range 1 to 100 pL.

12. The apparatus according to any one of the preceding claims, wherein the continuous liquid droplet generators are operable to produce liquid droplets at a frequency of more than 100 kHz.

13. The apparatus according to any one of the preceding claims, further comprising a microparticle-receiving means for receiving solid microparticles dispersed in a jet of liquid.

14. The apparatus according to any one of the preceding claims, further comprising a temperature regulator for controlling the temperature of liquid entering the liquid droplet generator and/or the temperature of liquid entering said nozzle.

15. The apparatus according to any one of the preceding claims, wherein the nozzle is arranged such that, in use, the jet is directed laterally so as to define a horizontal line or arc that passes below the liquid droplet generator.

16. The apparatus according to any one of claims 1 to 14, wherein the nozzle and liquid droplet generator are arranged such that the jet of the nozzle and a stream of the liquid droplets are both ejected substantially laterally through the gas such that they combine at a predefined point.

17. The apparatus according to any one of the preceding claims, wherein the outlets of adjacent liquid droplet generators are spaced-apart by between 5 and 25 mm, measured outlet centre-to-centre.

18. The apparatus according to any one of the preceding claims, wherein the continuous liquid droplet generator is positioned relative to the nozzle such that the distance of travel of a liquid droplet from the outlet of a liquid droplet generator to the jet is in the range 2 to 10 mm.

19. The apparatus according to any one of the preceding claims comprising a plurality of printing head arrangements

20. The apparatus according to claim 19, wherein the nozzles of the liquid droplet generators are spaced-apart at equal intervals.

21. The apparatus according to claim 19 or claim 20 wherein the plurality of printing head arrangements are arranged in parallel such that each of the liquid droplets are ejected in parallel and each of the jets are provided in parallel.

22. A process for producing solid microparticles, the process comprising:
providing a first liquid comprising a solute and a solvent, the solute comprising a biocompatible polymer, the concentration of polymer in the first liquid optionally being at least 10% w/v, ‘w’ being the weight of the polymer and ‘v’ being the volume of the solvent,
providing a continuous liquid droplet generator operable to generate liquid droplets by a continuous inkjet method,
providing a corresponding jet of a second liquid,
causing the liquid droplet generator to form liquid droplets of the first liquid,
passing the liquid droplets through a gas to contact the jet of the second liquid so as to cause the solvent to exit the droplets, thus forming solid microparticles,
wherein the solubility of the solvent in the second liquid is at least 5 g of solvent per 100 mL of second liquid, the solvent being substantially miscible with the second liquid.

23. The process according to claim 22, wherein the first liquid is a mixture that is prepared upstream of the liquid droplet generators by in-line mixing.

24. A process according to claim 22 or claim 23 wherein the first liquid comprises two components having a reaction half-life of two hours or less at standard temperature and pressure.

25. The process according to any one of claims 22 to 24, wherein the first liquid further comprises a target material which is desired to be encapsulated within the microparticles, the target material being incorporated in the first liquid as a particulate or in solution.

26. The process according to claim 25, wherein said target material comprises a pharmaceutically active agent or a precursor of a pharmaceutically active agent.

27. The process according to claim 25, wherein said target material comprises a pharmaceutically active agent or a precursor of a pharmaceutically active agent for treatment of a tumour, a central nervous system (CNS) condition, an ocular condition, an infection or an inflammatory condition.

28. The process according to claim 26 or claim 27, wherein said target material comprises a peptide, a hormone therapeutic, a chemotherapeutic or an immunosuppressant.

29. The process according to claim 25, wherein said target material comprises octreotide or a salt thereof, or ciclosporin A or a salt thereof.

30. The process according to any one of claims 25 to 29, wherein said target material comprises a plurality of nanoparticles.

31. The process according to claim 30, wherein said nanoparticles have a pharmaceutically active agent or a precursor of a pharmaceutically active agent covalently or non-covalently bound thereto.

32. The process according to any one of claims 22 to 31, wherein the continuous liquid droplet generator comprises at least one piezoelectric component operable to generate droplets.

33. The process according to claim 32 wherein the piezoelectric component is configured to generate an acoustic wave by piezo crystal distortion within an applied electric field such that the nozzle vibrates and the continuous flow is broken up into discrete droplets by a phenomenon known as ‘Rayleigh Instability’.

34. The process according to any one of claims 22 to 33, wherein the frequency of liquid droplet generation is more than 100 kHz.

35. The process according to any one of claims 22 to 34, wherein the jet of second liquid is generated by providing a continuous, pulseless flow of said second liquid and passing said flow of second liquid through a nozzle which causes a reduction in the cross-sectional area available for flow and thereby increases the flow velocity of the second liquid, said nozzle terminating in an orifice from which the jet of second liquid emerges.

36. The process according to any one of claims 22 to 35, wherein said jet of second liquid passes through a gas.

37. The process according to claim 35 or claim 36, wherein said jet of second liquid is not in contact with any wall or channel for at least part of its length.

38. The process according to claim 37, wherein said part of the length of the jet is not in contact with any wall or channel comprises a contact zone, said contact zone being the zone of the jet in which said liquid droplets make contact with said jet.

39. The process according to claim 38, wherein the liquid droplets pass through gas for a distance of less than 25 mm before contacting said jet of second liquid.

40. The process according to any one of claims 22 to 39, wherein said jet of second liquid flows at an angle greater than 0° and less than 90° relative to the direction of droplet ejection.

41. The process according to any one of claims 22 to 40, wherein the liquid droplet generator is positioned above the jet of second liquid and the liquid droplets are ejected downwards towards the jet of second liquid.

42. The process according to any one of claims 22 to 41, wherein said solvent is a biocompatible solvent.

43. The process according to any one of claims 22 to 42, wherein the second liquid comprises:
a mixture of water and an alcohol, optionally 10% to 20% v/v tertiary butanol in water; or
water and a water-soluble organic compound other than an alcohol.

44. The process according to any one of claims 22 to 43, wherein the polymer comprises a poly(lactide), a poly(glycolide), a polycaprolactone, a polyanhydride, a polyoxazoline, a polyphophazene and/or a co-polymer of lactic acid and glycolic acid or is any combination of said polymers or co-polymers.

45. The process according to any one of claims 22 to 43, wherein the polymer comprises Resomer RG752H, Purasorb PDL 02A, Purasorb PDL 02, Purasorb PDL 04, Purasorb PDL 04A, Purasorb PDL 05, Purasorb PDL 05A Purasorb PDL 20, Purasorb PDL 20A; Purasorb PG 20; Purasorb PDLG 5004, Purasorb PDLG 5002, Purasorb PDLG 7502, Purasorb PDLG 5004A, Purasorb PDLG 5002A, Resomer RG755S, Resomer RG503, Resomer RG502, Resomer RG503H, Resomer RG502H, Resomer RG752, PLGA-PEG, or any combination thereof.

46. The process according to any one of claims 22 to 45, wherein the process further comprises a step of collecting the solid microparticles by separating the solid microparticles from the second liquid.

47. A microparticle produced by the process according to any one of claims 22 to 46.

48. A pharmaceutical composition comprising the microparticle according to claim 47 and a pharmaceutically acceptable carrier, diluent, excipient, salt and/or solution.
